(19) United States
(12) Patent Application Publication
(10) Pub. No.: US 2026/0236781 A1
(43) Pub. Date: Aug. 13, 2026

(54) BIOMETRIC CONTRASTIVE LEARNING FOR DATA-EFFICIENT DEEP LEARNING FROM ELECTROCARDIOGRAPHIC IMAGES

(71) Applicant: Yale University, New Haven, CT (US)

(72) Inventors: Rohan Khera, New Haven, CT (US); Veer Sangha, New Haven, CT (US)

(21) Appl. No.: 19/155,244

(22) PCT Filed: Feb. 9, 2024
(86) PCT No.: PCT/US2024/015125

§ 371 Date: Aug. 9, 2025

Related U.S. Application Data
(60) Provisional application No. 63/484,426, filed on Feb. 10, 2023.

(51) Int. Cl. G06N 3/0895; A61B 5/346

(52) U.S. Cl. CPC G06N 3/0895; A61B 5/346

(57) ABSTRACT

Provided herein are methods of developing a deep learning model for detecting cardiovascular disorders from electrocardiographic images. The method includes pre-training the nodes of a deep neural network to detect homologies of electrocardiogram (ECG) signatures in distinct ECG images from a single person, and subsequently training the nodes on labeled ECG image examples of a cardiovascular disorder of interest. Also provided herein are methods of detecting cardiovascular disorders using the model.

### CROSS-REFERENCE TO RELATED APPLICATIONS

The present application claims priority under 35 U.S.C. § 119(e) to U.S. Provisional Patent Application No. 63/484,426, filed Feb. 10, 2023, which application is incorporated herein by reference in its entirety.

### BACKGROUND OF THE DISCLOSURE

Electrocardiography represents a ubiquitous tool currently used for the diagnosis of heart conduction and rhythm disorders. Deep learning has been successfully applied to automate both the detection of disorders that are commonly discernable by physicians from electrocardiograms (ECGs), as well as those that traditionally require more specialized imaging modalities such as echocardiography or cardiac magnetic resonance imaging scans. However, while most existing artificial intelligence (AI) tools to automatically analyze ECGs rely on raw signal data, most end users do not have access to ECG signals.

One attempt to address this includes an image-based approach that can interpretably diagnose disorders from ECG images. Despite the advantages of an image-based approach, algorithmic training and development require large, labeled datasets, while many clinical disorders have low prevalence, with few examples in any individual dataset to develop algorithms designed for those conditions. This low prevalence of clinical labels is a key challenge for development and generalizability of supervised learning approaches for ECG image models, particularly with respect to conditions that are rare or underdiagnosed in clinical settings.

Accordingly, there is a need in the art for articles and methods that improve on existing methods by detecting conditions with limited datasets. The present invention addresses this need.

### SUMMARY OF THE DISCLOSURE

In one aspect a method of developing a deep learning model for detecting cardiovascular disorders from electrocardiographic images includes pre-training the nodes of a deep neural network to detect homologies of electrocardiogram (ECG) signatures in distinct ECG images from a single person; and subsequently training the nodes on labeled ECG image examples of a cardiovascular disorder of interest. In some embodiments, the ECG images are format independent. In some embodiments, the format independent ECG images are selected from the group comprising a screenshot of at least a portion of an ECG, a photograph of at least a portion of an ECG, a saved image file of at least a portion of an ECG, and combinations thereof.

In some embodiments, the pre-training includes training the nodes to determine when distinct ECGs belong to the same person. In some embodiments, the training of the nodes to determine when distinct ECGs belong to the same person includes rewarding the model for identifying ECG images from the same individual as similar and penalizing the model for identifying ECG images from different individuals as similar. In some embodiments, the pre-training reduces a contrastive loss function. In some embodiments, the pre-training provides enhanced learning and detection of structural and functional abnormalities of the heart from any ECG image. In some embodiments, following the pre-training, the nodes recognize features that indicate whether distinct ECGs were obtained from a single person. In some embodiments, the features are hidden features.

In some embodiments, the model adapts to detection of the cardiovascular disorder of interest during the training on the labeled examples. In some embodiments, the pre-training is self-supervised and the subsequent training is supervised. In some embodiments, the method includes training the nodes to recognize individual leads of the ECG regardless of their location on the ECG image. In some embodiments, the method recognizes homologies of ECG signatures drawn from the same person.

In some embodiments, the labeled ECG image examples comprises a data set with a limited number of examples as compared to those for model development without pretraining procedures. In some embodiments, the cardiovascular disorder of interest is a rare structural or functional heart disease. In some embodiments, the model generates individualized phenotyping of disease in specific populations using a data set with a limited number of examples of patients with a disease as compared to those without pretraining procedure. In some embodiments, the model identifies markers of cardiovascular disease using the limited data set.

In some embodiments, the cardiovascular disorder of interest is a common disorder. In some embodiments, the model provides improved reliability for identification of physiological and pathological patterns on ECGs as compared to direct model development on the same data set.

In some embodiments, the method further includes detecting the cardiovascular disorder of interest by applying the model to ECG images from a subject. In some embodiments, the model detects cardiovascular disorders from ECG images in a decentralized manner.

### BRIEF DESCRIPTION OF THE DRAWINGS

FIG. 1 shows a schematic illustrating an overview of biometric contrastive learning (BCL).

FIGS. 2A-C show graphs illustrating various AUROC curves. (A) Ejection fraction <40.

(B) Atrial fibrillation. (C) Gender.

FIGS. 3A-C show graphs illustrating various AUPRC curves. (A) Ejection fraction <40. (B) Atrial fibrillation. (C) Gender.

FIGS. 4A-B show graphs illustrating AUROC and AUPRC gains. (A) Gain in AUROC using BCL versus other tech. (B) Gain in AUROC using BCL versus other tech.

FIG. 5 shows images illustrating model development and study design. Abbreviations: CMR, Cardiac Magnetic Resonance Imaging; HCM, Hypertrophic Cardiomyopathy; IVSd, End-diastolic Interventricular Septal Thickness.

FIGS. 6A-B show graphs illustrating model performance measures. (A) Receiver operating characteristic curves across image formats in a held-out test set. (B) Diagnostic odds ratios across age, gender, and race subgroups on standard format images in the held-out test set. Abbreviations: AUROC, area under receiver-operating characteristic curve.

FIGS. 7A-B show graphs illustrating distributions of interventricular septal thickness and left ventricular mass index in patients without hypertrophic cardiomyopathy. (A) Distribution of interventricular septal thickness in Yale New Haven Hospital patients without hypertrophic cardiomyopathy. (B) Distribution of left ventricular mass index in UK Biobank patients without hypertrophic cardiomyopathy.

FIGS. 8A-D show heatmaps illustrating gradient-weighted class activation mapping (Grad-CAMs) across electrocardiogram formats. (A) Standard format. (B) Two rhythm leads. (C) Standard shuffled format. (D) Alternate format. The heatmaps represent averages of the 25 positive cases with the most confident model predictions for HCM.

FIGS. 9A-C show a graph and heatmaps illustrating model performance in the UK Biobank. (A) Receiver Operating Characteristic Curves. (B-C) Examples of Gradient-weighted Class Activation Mapping (Grad-CAM) analysis of electrocardiograms from two individuals with HCM in UK Biobank. Abbreviations: AUROC, area under receiver-operating characteristic curve.

### DETAILED DESCRIPTION OF THE DISCLOSURE

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are methods of developing a deep learning model for detecting cardiovascular disorders from electrocardiographic images. In some embodiments, the method, also referred to herein as biometric contrastive learning (BCL), includes pre-training the nodes of a deep neural network to detect homologies of electrocardiogram (ECG) signatures in distinct ECG images from a single person, and subsequently training the nodes on labeled ECG image examples of a cardiovascular disorder of interest. The distinct ECG images may include any different ECG images, such as, but not limited to, different formats, different time points, modified ECG images, plots from different windows within a recording (e.g., a first plot from the first 5 seconds of a recording and another plot from a subsequent 5 seconds of the recording), combinations thereof, or any other suitable ECG images with at least one different property. In some embodiments, the modified ECG images include, but are not limited to, noised ECGs, different plotting variations, different background color, different font, different line width, any other suitable modification or variation to the ECG image, or a combination thereof. Additionally or alternatively, the ECG images may include images from any suitable number of leads, such as, but not limited to, at least 1 lead, at least 4 leads, at least 6 leads, at least 8 leads, at least 10 leads, at least 12 leads, between 1 and 12 leads, 1 lead, 6 leads, 12 leads, or any combination, sub-combination, range, or sub-range thereof.

In some embodiments, the pre-training includes training the nodes to determine when distinct ECGs belong to the same person. Such pre-training may include, but is not limited to, rewarding the model for identifying ECG images from the same individual as similar and penalizing the model for identifying ECG images from different individuals as similar. In some embodiments, the rewarding/penalizing is accomplished through a loss function, such as, but not limited to, NT-Xent loss, InfoNCE, triplet loss, contrastive loss, N-pair loss, or any other suitable loss function. For example, in one embodiment, the pre-training includes first providing and/or creating ECG images in different formats, where any two ECGs from the same person, in any two formats of image, are treated as a positive pair, and any pair of images from different individuals are treated as a negative pair. The ECG images in different formats are divided into batches that form a pre-training dataset, where each batch includes at least one positive pair embedded therein, with the remaining images being negative pairs. The model is then pre-trained on the pre-training dataset. In some embodiments, the ECG images of the pre-training dataset are unlabeled. Accordingly, in such embodiments, the pre-training is self-supervised.

As will be appreciated by those skilled in the art, although discussed herein with respect to a certain number of formats, batch sizes, and/or number of embedded positive pairs, the disclosure is not so limited and may include any suitable number of each. For example, although described herein as including four different image formats, the disclosure is not so limited and any embodiment may include more or less than four different formats (e.g., at least 2 different formats, at least 3 different formats, at least 4 different formats, at least 5 different formats, etc.). Similarly, although described herein as including a batch size of 16, the disclosure is not so limited and any embodiment may include more or less than 16 ECGs in a batch. Furthermore, although described herein as including a single positive pair in each batch, the disclosure is again not so limited and any embodiment may include more than one positive pair in each batch.

Following the pre-training, in some embodiments, the nodes recognize homologies of ECG signatures drawn from the same person and/or features that indicate whether distinct ECGs were obtained from a single person. In some embodiments, these features are hidden features. Additionally or alternatively, in some embodiments, the pre-training reduces a contrastive loss function, which depends on the cosine of similarity of embeddings of positive pairs in each batch compared to embeddings of negative pairs in the same batch. Furthermore, in some embodiments, the pre-training of the nodes according to the embodiments disclosed herein facilitates enhanced learning and detection of structural and functional abnormalities of the heart from any ECG image.

After the pre-training, the subsequent training includes supervised training to detect a feature or disorder of interest using a labeled dataset. The subsequent training includes any suitable amount of label fractions, including, but not limited to, 0.1%, 0.5%, 1%, 5%, 20%, 50%, and 100%, or combinations thereof. In some embodiments, the model developed with pre-training according to one or more of the embodiments disclosed herein provides improved reliability for identification of physiological and pathological patterns on ECGs, as compared to direct model development on the same data set. Due to the enhanced learning and/or improved reliability provided by the pre-training, the subsequent training may be performed using a data set with a limited or reduced number of labeled ECG image examples, as compared to data sets for model development without the pretraining procedures disclosed herein. In one embodiment, for example, the model is trained to generate individualized phenotyping of disease in specific populations using a data set with a limited number of examples of patients with a disease, as compared to those models without pretraining procedure. In another embodiment, the model identifies markers of cardiovascular disease using the limited data set.

As will be appreciated by those skilled in the art, the disclosure is not limited to features or disorders with limited datasets and includes training models for detection of a variety of different disorders, regardless of whether there is a limited dataset for the disorder (i.e., whether the disorder is common or rare). Accordingly, suitable disorders include, but are not limited to, common cardiovascular disorders, rare cardiovascular disorders (e.g., rare structural or functional heart disease), or any other suitable disorder detectable in ECGs. For example, in some embodiments, the subsequent training includes training the model to detect a cardiovascular disorder of interest using labeled examples from a limited or more robust dataset. Additionally or alternatively, in some embodiments, the method includes training the nodes to recognize individual leads of the ECG regardless of their location on the ECG image.

Also provided herein are computer-implemented methods of detecting features or disorders in a subject using an ECG image from the subject. In some embodiments, the method includes receiving ECG data from a subject, and applying the deep learning model according to any of the embodiments disclosed herein to the ECG data. In some embodiments, the model detects the feature or disease, such as a cardiovascular disorder, from ECG images in a decentralized (or federated) manner. In some embodiments, the ECG data for the subject includes format independent ECG images. Suitable format independent ECG images include, but are not limited to, a screenshot of at least a portion of an ECG, a photograph of at least a portion of an ECG, a saved image file of at least a portion of an ECG, or combinations thereof. In some embodiments, the method is automated and does not require human input in data extraction.

Suitable features and disorders that may be detected using the methods disclosed herein include, but are not limited to, any of the features and disorders disclosed herein, or any other suitable feature or disorder detectable in an ECG. The features and disorders may be common or rare. For example, in one embodiment, the method includes detecting hypertrophic cardiomyopathy (HCM), a genetic disease that affects 0.5% of the general population. Untreated HCM is associated with an increased risk of atrial fibrillation, stroke, and sudden cardiac death, especially among young and active individuals. While diagnosis and treatment can ameliorate symptoms, HCM remains underdiagnosed due to the need for advanced imaging or other diagnostic testing, which is resource intensive. Despite the rarity of HCM, the methods disclosed herein can detect hidden features of the disease undiscernible to the human eye and/or detect the disease through training with a limited dataset. Additionally, the methods disclosed herein can detect the disease using format independent ECG images and/or while recognizing individual leads of the ECG regardless of their location on the image.

Further provided herein are an apparatus for detecting features or disorders in a subject using ECG data from the subject and a computer readable storage medium storing computer-executable instructions for performing the method according to any of the embodiments disclosed herein. In some embodiments, the apparatus includes a processor, a memory unit, and a communication interface. The processor is connected to the memory unit and the communication interface, and the processor and memory are configured to implement the method.

The model according to one or more of the embodiments disclosed herein forms a superhuman reader of images and photos of ECGs that recognizes individual leads of the ECG like humans, regardless of their location of the page, as well as hidden features that are unique to an individual across multiple ECG recordings but are not discernable to humans. As such, the methods disclosed herein allow for a more efficient process of developing algorithms to detect hidden signatures of structural and functional heart disease that are not discernable to humans. Moreover, these models can be trained with a fraction of the data needed for current approaches, making them suitable for detecting and/or diagnosing rare structural and functional heart disease using photos of ECGs. In addition, when trained with the pre-training methods disclosed herein, diagnosis models for more relatively common disorders are more generalizable and rely more on physiological patterns. Furthermore, algorithms trained according to the models disclosed herein can be used by either end users, such as patients and clinicians directly using applications directly on their smartphone devices, or by hospitals in their ECG image repositories for the detection of disorders.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

This Example describes a novel, few-shot, deep learning model development strategy—biometric contrastive learning (BCL)—in which the models are first trained to detect homologies of ECG features belonging to the same person, allowing for enhanced learning and detection of structural and functional abnormalities of the heart from any ECG image. By developing a model that is designed to learn that two distinct ECGs belong to the same person, the approach discussed herein builds a model that already recognizes key hidden feature that make two ECGs of a single person similar. This early training process can then be trained on a small number of labeled examples of any disease of interest, and the model is able to adapt to detection of the new disorder.

### Methods

### Overview

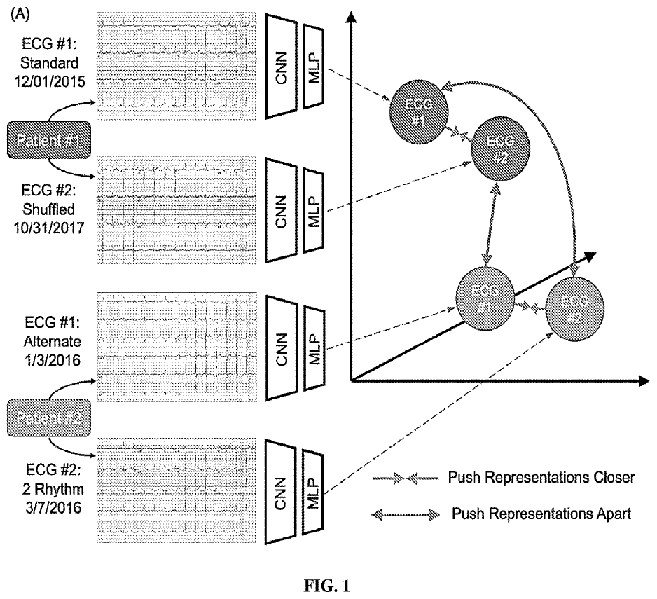

BCL used a convolutional neural network (CNN) backbone to build representations of ECGs specific to individuals (FIG. 1). During training, the model learned the elements of an ECG image that are consistent for a person. The model was rewarded for identifying ECG images from the same individual as similar and penalized for identifying ECG images from different individuals as similar.

### Positive and Negative Views

To develop this model, we created ECG images in different formats. ECGs were plotted in one of four formats—1) a standard format with 4 columns with 2.5 seconds of recordings and a rhythm lead on the bottom, 2) a format with a second rhythm lead, 3) a shuffled format with precordial leads on the right side of the image and limb leads on the left side, and 4) an alternate format with two columns each containing 5 seconds of recordings from 6 leads.

Any two ECGs from the same person, in any two formats of image, rotated between-10 and 10 degrees were treated as a positive pair. Any pair of images from different individuals were treated as a negative pair.

### Alternative Approaches

In addition to BCL, we tested two traditional approaches for model pretraining. The first of these was random weight initialization. The second, was the classic SimCLR contrastive pretraining method that has been popular in image processing such as those to identify objects in real-world photos. The latter approach, developed by Google, uses cropped and flipped parts of an image to identify the parts that are derived from the same image.

### Model Training

We chose an Efficientnet-B3 CNN architecture for our encoder, following the demonstrated effectiveness of the model on other ECG image classification tasks. However, the method is not restricted to this CNN architecture. ECG images were resampled to 300×300 pixels prior to being input into the model and were converted to greyscale.

Our goal during pretraining was to minimize the contrastive loss function, which depended on the cosine similarity of embeddings of positive pairs in each batch compared to embeddings of negative pairs in the same batch. We used batch sizes of 16, with each ECG in the batch having one positive pair and 14 negative pairs, and accumulated gradients over 16 batches before updating the model.

Our encoder had an output dimension of 1,536. We used a 2-layer multilayer perceptron (MLP) in pretraining which projected the 1,536-dimensional output of the encoder into a 128-dimensional space. We used an Adam optimizer with learning rate of 1×10⁻⁵ for pretraining.

### Pretraining Dataset for BCL

We pretrained our model using ECGs acquired between 2000 and 2015 without labels from the Yale New Haven Health System. 78,288 patients with a pair of ECGs between 5 and 1000 days apart during this time were identified. For each patient, one pair of ECGs—the one with the smallest time difference at least 5 days apart—was chosen. For each pair of ECGs, 6 unique pairs were created, corresponding to potential combinations where ECGs were in two different out of the 4 total image formats. In total, there were 469,728 total pairs of ECGs. During pretraining, we ensured that per batch there were no more than one pair of ECGs from any given patient.

### Downstream Tasks

We performed downstream fine tuning and evaluation of our pretrained encoder on three tasks. These were the detection of gender, atrial fibrillation (AFib), and left ventricular systolic dysfunction (LVSD), defined as ejection fraction (EF)<40%. For all three of these tasks, we fine-tuned our encoder on 0.1%, 0.5%, 1%, 5%, 20%, 50%, and 100% label fractions of these tasks.

### Results

We demonstrate that pursuing a BCL pretraining consistently build classification models that outperform models developed using other approaches, namely random initialization or pretraining with the SimCLR approach. These patterns are particularly relevant when the volume of labeled data is low. Therefore, while at 100% of the data available, the three strategies (BCL, random, simCLR) were comparable for the 3 tasks of identifying gender, atrial fibrillation, and LVSD, on both AUROC and AUPRC, the performance metrics we used to evaluate models.

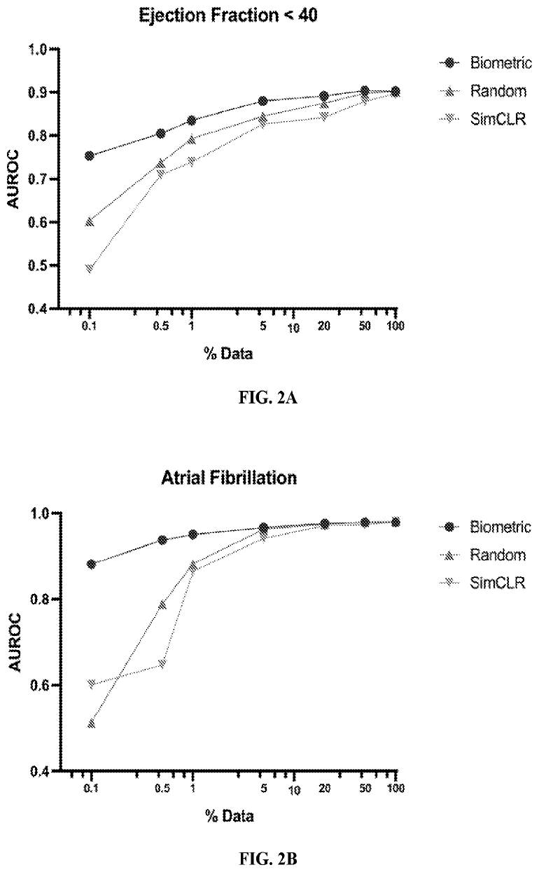

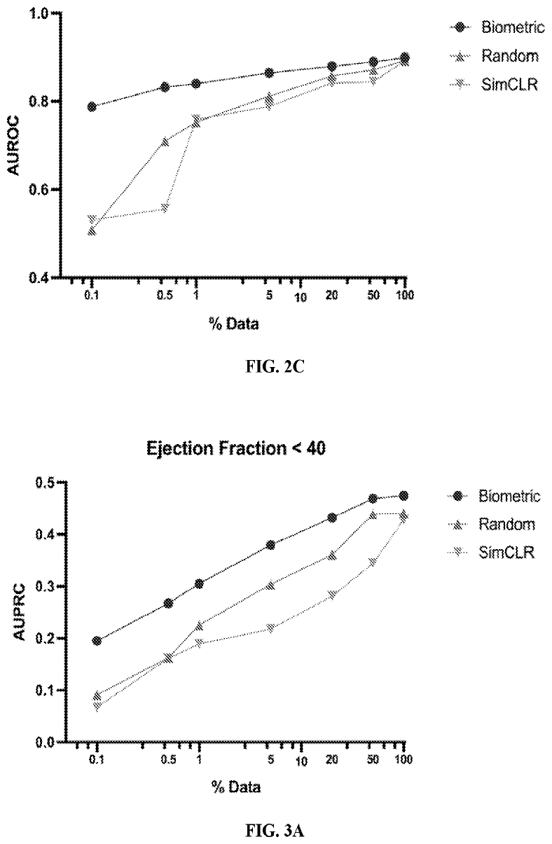

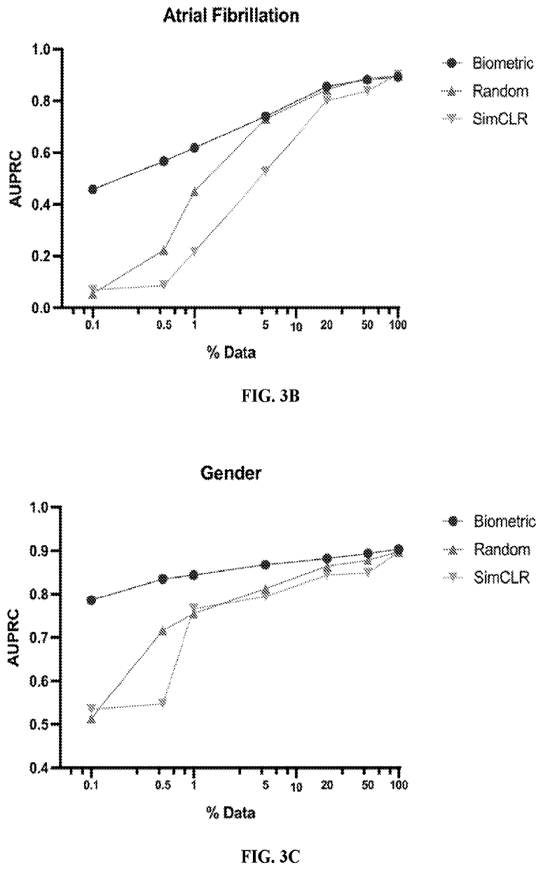

However, across the three tasks, BCL demonstrated equivalent performance for both AUROC (FIGS. 2A-C) and AUPRC (FIGS. 3A-C) at 50% of the data available as it did with 100% of the data, even though the other models initialized with random weights and using a SimCLR approach suffered drop-offs in performance.

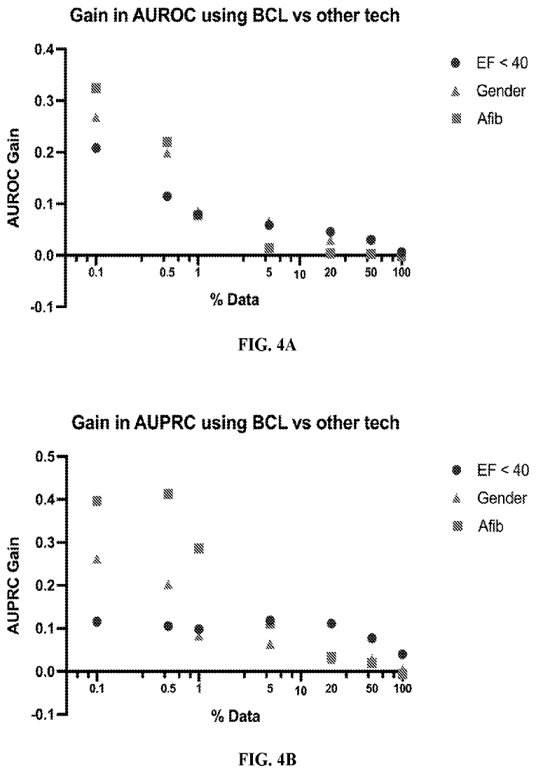

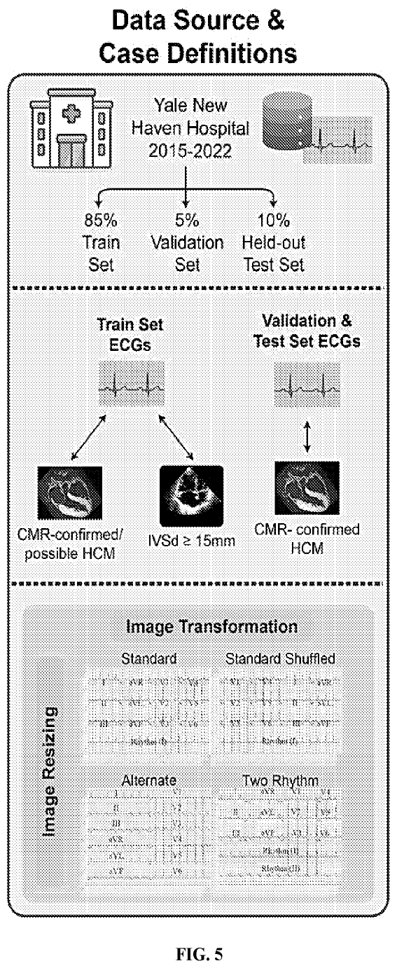

As the quantity of data available progressively decreased, models trained using all three strategies saw lower performance due to a scarcity of labelled training data, but BCL consistently outperformed other methods, with the difference in performance between BCL and other methods growing as labelled data became more scarce (FIGS. 2A-3C). On models trained with 1% of the available data, AUROC for models trained with BCL on the tasks of detecting EF<40, AFib, and gender was 0.84, 0.95, and 0.84 respectively, while AUROC for randomly initialized models was 0.79, 0.88, and 0.75 respectively, and AUROC for models trained with SimCLR was 0.74, 0.86, and 0.76 respectively. This corresponded to an average gain in AUROC of 0.08 between BCL and the other two methods across tested applications (FIG. 4A).

On models trained with 1% of the data available, AUPRC for models trained with BCL on the tasks of detecting EF<40, AFib, and gender was 0.30, 0.62, and 0.84 respectively, while AUPRC for randomly initialized models was 0.22, 0.45, and 0.76 respectively, and AUPRC for models trained with SimCLR was 0.19, 0.22, and 0.77 respectively. This corresponded to an average gain in AUPRC of 0.16 between BCL and the other two methods across tested applications (FIG. 4B).

On models trained with the smallest fraction of data available, 0.1%, AUROC for models trained with BCL on the tasks of detecting EF<40, AFib, and Gender was 0.74, 0.88, and 0.79 respectively, and AUPRC was 0.19, 0.46, and 0.79. AUROC for randomly initialized models was 0.57, 0.51, and 0.51 respectively, and AUPRC was 0.09, 0.05, and 0.51. Finally, AUROC for models trained with SimCLR was 0.50, 0.60, and 0.53 respectively, and AUPRC was 0.07, 0.07, and 0.53. This corresponded to an average gain in AUROC of 0.27 and AUPRC of 0.26 between BCL and the other two methods across tested applications (FIGS. 4A-B).

### Conclusion

Our novel biometric pretraining framework through a focus on building models to identify shared features of ECGs drawn from the same person at different times, but plotted in different layouts and different augmentations, allows label-efficient learning on ECG images for novel tasks. Therefore, we are able to use unabeled data to achieve large gains in the model development process for applications with sparsely labeled datasets or when clinical disorders are rare. For these applications, while the model is not explicitly trained for any clinical diagnosis identification task in our pretraining process, it learns deeper representations from ECGs across different layouts, such that a format-independent deep learning model for ECG images can be trained with few positive and negative examples. The approach consistently outperforms the current standard of using out-of-box models with random initializations or those pretrained using other contrastive pretraining approaches, such as the standard SimCLR. The ECG-adapted, novel strategy that leverages hidden homologies of ECGs from the same person represents a significant advance in layout/format-independent deep learning for the detection of structural and functional heart disease directly from ECG images.

### Example 2

### Abstract

Background: Hypertrophic cardiomyopathy (HCM) affects 1 in every 200 individuals and is the leading cause of sudden cardiac death in young adults. HCM can be identified using an electrocardiogram (ECG) raw voltage data and deep learning approaches, but their point-of-care application is limited by the inaccessibility of these signal data. We developed a deep learning-based approach that overcomes this limitation and detects HCM from images of 12-lead ECGs across layouts.

Methods: We identified ECGs from patients with HCM features present on cardiac magnetic resonance imaging (CMR) or those within 30 days of an echocardiogram documenting thickened interventricular septum (end-diastolic interventricular septum thickness >15 mm). Patients with CMR-confirmed HCM were considered as cases during the final model evaluation. The model was validated within clinical settings at YNHH and externally on ECG images from the prospective, population-based UK Biobank cohort. We localized class-discriminating signals in ECG images using gradient-weighted class activation mapping.

Results: Overall, 124,553 ECGs from 66,987 individuals (HCM cases and controls) were used for model development. The model demonstrated high discrimination for HCM across various ECG image formats and calibrations in internal validation (area under receiving operation characteristics [AUROC] 0.96) and external sets of ECG images from UK Biobank (AUROC 0.94). A positive screen for HCM was associated with a 100-fold higher odds of CMR-confirmed HCM (OR 102.4, 95% Confidence Interval, 57.4-182.6) in the held-out set. Class-discriminative patterns localized to the anterior and lateral leads (V4-V5).

Conclusions: We developed and externally validated a deep learning model that identifies HCM from ECG images with excellent discrimination. This approach represents an automated, efficient, and accessible screening strategy for HCM.

### INTRODUCTION

While hypertrophic cardiomyopathy (HCM) is among the leading causes of sudden cardiac death, scalable solutions for screening for the disease have remained elusive. HCM is a genetically determined disease that affects up to 1 in every 200 people globally. An early diagnosis of HCM can enable regular healthcare follow-up, rigorous cardiovascular risk management, and timely initiation of highly effective risk-reducing therapies. The diagnosis of HCM has relied on cardiac imaging, such as echocardiography and cardiac magnetic resonance imaging (CMR). However, given the requirement of expensive technology and extensive clinical expertise in deploying and interpreting these modalities, using echocardiography or CMR as a screening strategy is not feasible.

Given the inaccessibility of advanced cardiac imaging, deep learning or artificial intelligence (AI)-enhanced interpretation of electrocardiograms (AI-ECG) has been proposed as an alternative for the early detection of HCM. While ECG abnormalities, such as prominent Q waves, repolarization changes, left axis deviation, or giant negative T waves, can be apparent in over 90% of patients with the disease, these changes are not specific to HCM. Deep learning-based approaches, such as convolutional neural networks (CNNs) can leverage HCM-specific pathological signatures to identify people the disease using clinical ECGs. Current models, however, use raw ECG voltage data as the inputs, which are often stored in vendor-specific formats and rarely accessible to clinicians at the point-of-care. Moreover, for identifying patients with HCM in development cohorts, a combination of diagnosis codes and other administrative data sources, such as visits to the HCM clinic, have been used. Such administrative-code-based phenotyping of diseases in the electronic health record is prone to misclassification and variability due to vast differences in health-system-specific coding practices for HCM. Therefore, there is an unmet need for the development of models that use ubiquitous and interoperable data formats for disease diagnosis and rely on objective imaging-based biological features for defining the presence of disease to enable AI-ECG's use as a practical, generalizable, and scalable screening modality for HCM.

In this study, we report the development and validation of a deep learning-based approach for identifying CMR-confirmed HCM using images of clinical 12-lead ECGs.

### Methods

The Yale Institutional Review Board approved the study protocol and waived the need for informed consent as the study represents a secondary analysis of existing data. Patients who opted out of research studies at the Yale New Haven Hospital (YNHH) were not included. An online version of the model is publicly available for research use at https://www.cards-lab.org/ecgvision-hcm. This web application represents a prototype of the eventual application of the model, with instructions for required image standards and a version that demonstrates an automated image standardization pipeline.

### Data Source and Study Population

We used 12-lead ECG signal waveform data collected during the clinical care of patients at the YNHH between 2012 and 2021. These ECGs were recorded as standard 12-lead recordings sampled at a frequency of 500 Hz for 10 seconds. These were recorded on multiple different machines, primarily the Philips PageWriter and GE MAC machines.

We identified the earliest MRI reports for 1,061 patients containing any mention of HCM. Each of these reports was manually reviewed by three cardiologists, and 904 people were identified as having confirmed or possible HCM. Of these, 779 reports included a confirmed HCM diagnosis and 125 were identified as possible HCM, defined as the inclusion of HCM as one of the reported differential diagnoses, or the presence of features potentially suggestive, but not conclusive for HCM. The data from these patients were split into mutually exclusive training, validation, and test sets in an 85:5:10 ratio. Only MRI-confirmed HCM cases were retained in validation and testing, while confirmed and possible HCM cases were included in the training cohort. For each of these patients, all ECGs recorded up to a year before the MRI, and any time after the MRI were considered, except for those ECGs after a septal reduction procedure, including alcohol septal ablation or ventricular myectomy. To further augment the training cohort, we incorporated ECGs from patients whose transthoracic echocardiograms (TTE) demonstrated severe ventricular hypertrophy (LVH), defined by interventricular septal thickness in diastole (IVSd) of greater than 15 mm. We posited that these may represent individuals with possible HCM. ECGs performed within 30 days before or after a TTE demonstrating severe LVH were included as cases in the training set, but were not considered cases in the test set, which only included CMR-confirmed HCM. For patients with more than one recorded ECG filling these criteria, a maximum of five most recent ECGs were used to create cohorts to avoid overrepresenting patients undergoing frequent ECGs.

To derive a control cohort for the training set, we identified ECGs recorded within 30 days of a TTE in a cohort of patients who did not have a diagnosis code recorded for HCM (Table 1) and were not in the cohort of patients with possible positive HCM cases based on MRI reports or IVSd values. Thus, patients with any ICD code for HCM (Table 1), any mention of HCM in cardiac MRI reports, or any TTE with IVSd >15 mm were not included in the control cohort. We randomly sampled control ECGs so the train set had a 10% prevalence of HCM ECGs to allow the model to learn signatures of HCM on ECG successfully.

TABLE 1
International classification of disease tenth revision (ICD-10) codes for hypertrophic
cardiomyopathy and conditions indicating cardiomyopathies.

| Condition | International Classification of Disease Tenth Revision (ICD-10) Codes |
|---|---|
| Hypertrophic | ‘I42.1’, ‘I42.2’ |
| Cardiomyopathy | |
| Heart Failure | ‘I50’, ‘I50.1’, ‘I50.2’, ‘I50.20’, ‘I50.21’, ‘I50.22’, ‘I50.23’, ‘I50.3’, ‘I50.30’, ‘I50.31’, |
| | ‘I50.32’, ‘I50.33’, ‘I50.4’, ‘I50.40’, ‘I50.41’, ‘I50.42’, ‘I50.43’, ‘I50.8’, ‘I50.81’, |
| | ‘I50.810’, ‘I50.811’, ‘I50.812’, ‘I50.813’, ‘I50.814’, ‘I50.82’, ‘I50.83’, ‘I50.84’, |
| | ‘I50.89’, ‘I50.9’ |
| Conditions Indicating | ‘I21.01’, ‘I21.02’, ‘I21.09’, ‘I21.11’, ‘I21.19’, ‘I21.21’, ‘I21.29’, ‘I21.3’, ‘I21.4’, |
| Cardiomyopathies | ‘I21.9’, ‘I22.0’, ‘I22.1’, ‘I22.2’, ‘I22.8’, ‘I22.9’, ‘I23.0’, ‘I23.1’, ‘I23.2’, ‘I23.3’, ‘I23.4’, |
| (considered in patients | ‘I23.5’, ‘I23.6’, ‘I23.7’, ‘I23.8’, ‘I24.0’, ‘I24.8’, ‘I24.9’, ‘I25.10’, ‘I25.110’, ‘I25.111’, |
| with inpatient | ‘I25.118’, ‘I25.119’, ‘I25.2’, ‘I25.5’, ‘I25.5’, ‘I25.6’, ‘I25.700’, ‘I25.701’, ‘I25.708’, |
| hospitalization with a | ‘I25.709’, 125.710’, ‘I25.711’, ‘I25.718’, ‘I25.719’, ‘I25.720’, ‘I25.721’, ‘I25.728’, |
| heart failure diagnosis) | ‘I25.729’, ‘I25.730’, ‘I25.731’, ‘I25.738’, ‘I25.739’, ‘I25.750’, ‘I25.751’, ‘I25.758’, |
| | ‘I25.759’, ‘I25.760’, ‘I25.761’, ‘I25.768’, ‘I25.769’, ‘I25.790’, ‘I25.791’, ‘I25.798’, |
| | ‘I25.799’, ‘I25.810’, ‘I25.811’, ‘I25.812’, ‘I25.82’, ‘I25.83’, ‘I25.84’, ‘I25.89’, ‘I25.9’, |
| | ‘T82.855A’, ‘T82.01XA’, ‘T82.01XD’, ‘T82.01XS’, ‘T82.02XA’, ‘T82.02XD’, |
| | ‘T82.02XS’, ‘T82.03XA’, ‘T82.03XD’, ‘T82.03XS’, ‘T82.09XA’, ‘T82.09XD’, |
| | ‘T82.09XS’, ‘T82.221A’, ‘T82.221D’, ‘T82.221S’, ‘T82.222A’, ‘T82.222D’, |
| | ‘T82.222S’, ‘T82.223A’, ‘T82.223D’, ‘T82.223S’, ‘T82.228A’, ‘T82.228D’, |
| | ‘T82.228S’, ‘T82.6XXA’, ‘T82.6XXD’, ‘T82.6XXS’, ‘Z95.2’, ‘Z95.2’, ‘Z95.3’, |
| | ‘Z95.3’, ‘Z95.4’, ‘Z95.4’, ‘I05.8’, ‘I05.9’, ‘I06.8’, ‘I06.9’, ‘I07.8’, ‘I07.9’, ‘I08.0’, |
| | ‘I08.1’, ‘I08.2’, ‘I08.3’, ‘I08.8’, ‘I08.9’, ‘I09.1’, ‘I34.0’, ‘I34.1’, ‘I34.2’, ‘I34.8’, ‘I34.9’, |
| | ‘I35.0’, ‘I35.1’, ‘I35.2’, ‘I35.8’, ‘I35.9’, ‘I36.0’, ‘I36.1’, ‘I36.2’, ‘I36.8’, ‘I36.9’, ‘I37.0’, |
| | ‘I37.1’, ‘I37.2’, ‘I37.8’, ‘I37.9’, ‘I38.’, ‘I39.’, ‘Q22.0’, ‘Q22.1’, ‘Q22.2’, ‘Q22.3’, |
| | ‘Q22.8’, ‘Q22.9’, ‘Q23.0’, ‘Q23.1’, ‘Q23.8’, ‘Q23.9’, ‘Q25.3’, ‘Q64.2’, ‘I47.0’, ‘I47.1’, |
| | ‘I47.2’, ‘I47.9’, ‘I48.0’, ‘I48.1’, ‘I48.11’, ‘I48.19’, ‘I48.2’, ‘I48.20’, ‘I48.21’, ‘I48.3’, |
| | ‘I48.4’, ‘I48.91’, ‘I48.92’, ‘I49.01’, ‘I49.02’, ‘I49.1’, ‘I49.2’, ‘I49.3’, ‘I49.40’, ‘I49.49’, |
| | ‘I49.5’, ‘I49.8’, ‘I49.9’, ‘Z8.6.79’, ‘I11.0’, ‘I11.0’, ‘I11.9’, ‘I12.0’, ‘I12.0’, ‘I12.9’, |
| | ‘I12.9’, ‘I13.0’, ‘I13.0’, ‘I13.0’, ‘I13.10’, ‘I13.10’, ‘I13.11’, ‘I13.11’, ‘I13.2’, ‘I13.2’, |
| | ‘I13.2’, ‘I16.0’, ‘I16.1’, ‘I16.9’, ‘I67.4’, ‘I67.4’, ‘H35.031’, ‘H35.031’, ‘H35.032’, |
| | ‘H35.032’, ‘H35.033’, ‘H35.033’, ‘H35.039’, ‘I42.6’, ‘I42.6’, ‘K29.20’, ‘K29.21’, |
| | ‘K70.40’, ‘K70.41’, ‘K70.0’, ‘K70.2’, ‘K70.30’, ‘K70.31’, ‘K70.9’, ‘K85.2’, ‘K85.20’, |
| | ‘K85.21’, ‘K85.22’, ‘K86.0’, ‘K29.21’, ‘T40.5X1A’, ‘T40.5X2A’, ‘T40.5X3A’, |
| | ‘T40.5X4A’, ‘T51.8X1A’, ‘T51.8X2A’, ‘T51.8X3A’, ‘T51.8X4A’, ‘T51.91XA’, |
| | ‘T51.92XA’, ‘T51.93XA’, ‘T51.94XA’, ‘T40.5X1A’, ‘T51.8X1A’, ‘T51.91XA’, |
| | ‘T40.5X2A’, ‘T51.8X2A’, ‘T51.92XA’, ‘T40.5X3A’, ‘T51.8X3A’, ‘T51.93XA’, |
| | ‘T40.5X4A’, ‘T51.8X4A’, ‘T51.94XA’, ‘Y90.2’, ‘Y90.3’, ‘Y90.4’, ‘Y90.5’, ‘Y90.6’, |
| | ‘Y90.7’, ‘Y90.8’, ‘Y90.9’, ‘Z71.41’, ‘Z71.42’, ‘Z63.72’, ‘K70.10’, ‘K70.11’, |
| | ‘T40.5X1A‘ ‘T40.5X2A’, ‘T40.5X3A’, ‘T40.5X4A’, ‘T51.8X1A’, ‘T51.8X2A’, |
| | ‘T51.8X3A’, ‘T51.8X4A’, ‘T51.91XA’, ‘T51.92XA’, ‘T51.93XA’, ‘T51.94XA’ |
| | ‘T40.5X5A’, ‘T40.5X6A’, ‘T40.5X1D’, ‘T40.5X2D’, ‘T40.5X3D’, ‘T40.5X4D’, |
| | ‘T51.8X1D’, ‘T51.8X2D’, ‘T51.8X3D’, ‘T51.8X4D’, ‘T51.91XD’, ‘T51.92XD’, |
| | ‘T51.93XD’, ‘T51.94XD’, ‘T40.5X5D’, ‘T40.5X6D’, ‘T40.5X1S’, ‘T40.5X2S’, |
| | ‘T40.5X3S’, ‘T40.5X4S’, ‘T40.5X5S’, ‘T40.5X6S’, ‘T51.8X1S’, ‘T51.8X2S’, |
| | ‘T51.8X3S’, ‘T51.8X4S’, ‘T51.91XS’, ‘T51.92XS’, ‘T51.93XS’, ‘T51.94XS’, |
| | ‘Q86.0’, ‘F10.150’, ‘F10.151’, ‘F10.159’, ‘F10.250’, ‘F10.251’, ‘F10.259’, ‘F10.950’, |
| | ‘F10.951’, ‘F10.959’, ‘F14.150’, ‘F14.151’, ‘F14.159’, ‘F14.250’, ‘F14.251’, |
| | ‘F14.259’, ‘F14.950’, ‘F14.951’, ‘F14.959’, ‘F10.180’, ‘F10.280’, ‘F10.980’, |
| | ‘F14.180’, ‘F14.280’, ‘F14.980’, ‘T40.5X2A’, ‘T51.8X2A’, ‘T51.92XA’, ‘F10.10’, |
| | ‘F10.120’, ‘F10.121’, ‘F10.129’, ‘F10.130’, ‘F10.131’, ‘F10.132’, ‘F10.139’, ‘F10.14’, |
| | ‘F10.150’, ‘F10.151’, ‘F10.159’, ‘F10.180’, ‘F10.181’, ‘F10.182’, ‘F10.188’, ‘F10.19’, |
| | ‘F10.20’, ‘F10.220’, ‘F10.221’, ‘F10.229’, ‘F10.230’, ‘F10.231’, ‘F10.232’, ‘F10.239’, |
| | ‘F10.24’, ‘F10.250’, ‘F10.251’, ‘F10.259’, ‘F10.26’, ‘F10.27’, ‘F10.280’, ‘F10.281’, |
| | ‘F10.282’, ‘F10.288’, ‘F10.29’, ‘F10.920’, ‘F10.921’, ‘F10.929’, ‘F10.930’, ‘F10.931’, |
| | ‘F10.932’, ‘F10.939’, ‘F10.94’, ‘F10.950’, ‘F10.951’, ‘F10.959’, ‘F10.96’, ‘F10.97’, |
| | ‘F10.980’, ‘F10.981’, ‘F10.982’, ‘F10.988’, ‘F10.99’, ‘G31.2’, ‘G62.1’, ‘K29.20’, |
| | ‘K29.21’, ‘K70.0’, ‘K70.10’, ‘K70.11’, ‘K70.2’, ‘K70.30’, ‘K70.31’, ‘K70.40’, |
| | ‘K70.41’, ‘K70.9’, ‘F14.10’, ‘F14.120’, ‘F14.121’, ‘F14.122’, ‘F14.129’, ‘F14.13’, |
| | ‘F14.14’, ‘F14.150’, ‘F14.151’, ‘F14.159’, ‘F14.180’, ‘F14.181’, ‘F14.182’, ‘F14.188’, |
| | ‘F14.19’, ‘F14.20’, ‘F14.220’, ‘F14.221’, ‘F14.222’, ‘F14.229’, ‘F14.23’, ‘F14.24’, |
| | ‘F14.250’, ‘F14.251’, ‘F14.259’, ‘F14.280’, ‘F14.281’, ‘F14.282’, ‘F14.288’, ‘F14.29’, |
| | ‘F14.90’, ‘F14.920’, ‘F14.921’, ‘F14.922’, ‘F14.929’, ‘F14.93’, ‘F14.94’, ‘F14.950’, |
| | ‘F14.951’, ‘F14.959’, ‘F14.980’, ‘F14.981’, ‘F14.982’, ‘F14.988’, ‘F14.99’, |
| | ‘T40.5X1A’, ‘T40.5X2A’, ‘T40.5X3A’, ‘T40.5X4A’, ‘T40.5X5A’, ‘F10.11’, ‘F10.21’, |
| | ‘F14.11’, ‘F14.21’, ‘T40.5X2D’, ‘T51.8X2D’, ‘T51.92XD’, ‘T40.5X1D’, ‘T40.5X2D’, |
| | ‘T40.5X3D’, ‘T40.5X4D’, ‘T40.5X5D’, ‘T40.5X1S’, ‘T40.5X2S’, ‘T40.5X3S’, |
| | ‘T40.5X4S’, ‘T40.5X5S’, ‘T51.8X2S’, ‘T51.92XS’, ‘G31.2’, ‘K70.41’, ‘G62.1’, |
| | ‘G72.1’, ‘O99.314’, ‘O99.315’, ‘O99.310’, ‘O99.311’, ‘O99.312’, ‘O99.313’, ‘R78.0’, |
| | ‘R78.2’, ‘A36.81’, ‘B33.24’, ‘I42.0’, ‘I42.5’, ‘I42.7’, ‘I42.9’, ‘I43.’, ‘O90.3’, ‘A38.1’, |
| | ‘A39.50’, ‘A39.52’, ‘B26.82’, ‘B33.20’, ‘B33.22’, ‘B58.81’, ‘I01.2’, ‘I09.0’, ‘I40.0’, |
| | ‘I40.1’, ‘I40.8’, ‘I40.9’, ‘I41.’, ‘I42.3’, ‘I42.4’, ‘I42.8’, ‘I51.4’, ‘J10.82’, ‘J11.82’, |
| | ‘I51.81’, ‘E74.00’, ‘E74.01’, ‘E74.02’, ‘E74.03’, ‘E74.04’, ‘E74.09’, ‘E88.49’, ‘E88.81’, |
| | ‘E88.89’, ‘E88.9’, ‘O90.3’, ‘A36.81’, ‘B33.24’, ‘I42.7’, ‘E83.110’, ‘E83.118’, ‘E83.19’ |

Note:
Conditions indicating cardiomyopathies were only considered as present, if the patients also had an inpatient hospitalization with a heart failure diagnosis code.

To identify a control cohort for the validation and test sets, we used ECGs from any patients who did not have any diagnosis code suggestive of conditions causing cardiomyopathy, along with an inpatient hospitalization for heart failure (Table 1). These patients were also not present in the cohort of patients with confirmed or possible HCM cases based on MRI reports. Since HCM more commonly affects men and is diagnosed in the younger population, it is important to ensure that the model identifies the pathological signature of the HCM and does not rely on age- and sex-based ECG features for the HCM prediction. Thus, the validation and test controls were age- and sex-matched to HCM ECGs at a 10:1 prevalence. We identified 10 control ECGs from patients within 5 years of age, and of the same sex, as each HCM case ECG. We ensured no patient overlap between training, validation, and test sets.

### Image Generation

We generated ECG images to recapitulate the variation in ECG layouts in a real-world setting. Our approach to image plotting has been previously described and represents the processing steps of ECG machines to convert acquired waveform data to printed outputs (Daskalov I K, Dotsinsky I A, Christov I I. Developments in ECG acquisition, preprocessing, parameter measurement, and recording. IEEE Eng Med Biol Mag. 1998; 17:50-58; Blanco-Velasco M, Weng B, Barner K E. ECG signal denoising and baseline wander correction based on the empirical mode decomposition. Comput Biol Med. 2008; 38:1-13). Briefly, all ECGs were analyzed to determine whether they had 10 seconds of continuous recordings across all 12 leads. The 10-second samples were preprocessed with a one-second median filter, subtracted from the original waveform to remove baseline drift in each lead. The process of converting ECG signals to images was independent of model development, ensuring that the model did not learn any aspects of the processing that generated images from the signals.

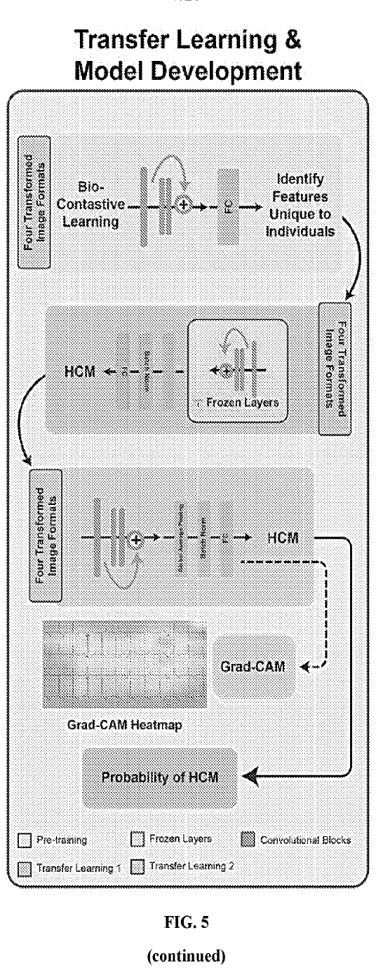

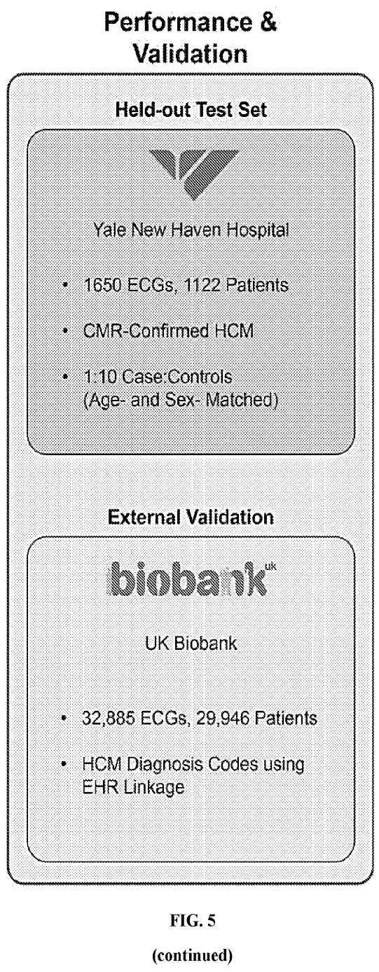

ECG signals were transformed into ECG images using the Python library ecg-plot. Images were generated with a calibration of 10 mm/mV, which is standard for printed ECGs in most real-world settings. Using the Python Image Library (PIL v9.2.0), we converted all images to greyscale, followed by down-sampling to 300×300 pixels regardless of their original resolution. Given that real-world ECG images may vary in the layout of leads, we created a dataset with four different plotting schemes for each signal waveform recording (FIG. 5). The first format was based on the standard printed ECG format in the United States. This format consisted of four columns printed sequentially, each containing 2.5-second intervals from three leads. The full 10-second recording of the lead I signal was included as the rhythm strip. The second format, a two-rhythm format, added lead II as an additional rhythm strip to the standard format. The third layout was the alternate format, which consisted of two columns, the first with six simultaneous 5-second recordings from the limb leads and the second with six simultaneous 5-second recordings from the precordial leads, without a corresponding rhythm lead. The fourth format was a shuffled format, which had precordial leads in the first two columns and limb leads in the third and fourth. All images were rotated a random amount between-10 and 10 degrees before being input into the model to mimic variations seen in uploaded ECGs and to aid in the prevention of overfitting.

### Model Architecture and Training

We built a convolutional neural network model based on the EfficientNet-B3 architecture. The EfficientNet-B3 model requires images to be sampled at 300×300 square pixels, includes 384 layers, and has over 10 million trainable parameters. To allow label-efficient model development, we initialized the model using weights from a pretrained EfficientNet-B3 model that leveraged a novel self-supervised biometric contrastive learning approach, wherein the model was trained to identify individual patient-specific patterns in ECGs regardless of their interpretation. None of the ECGs on the self-supervised pretraining task represented individuals in the model development. For training, we first unfroze the last four layers and trained the model with a learning rate of 0.01 for 2 epochs. Then, we unfroze all layers and trained the model with a learning rate of 5×10⁻⁶ for 6 epochs. We used an Adam optimizer, gradient clipping, and a minibatch size of 64 throughout training. The optimizer and learning rates were chosen after hyperparameter optimization. For both stages of training the model, we stopped training when validation loss did not improve in 3 consecutive epochs. A custom class-balanced loss function (weighted binary cross-entropy) based on the effective number of samples was used given that the case and control labels were not equally balanced.

Evaluation of Hypertrophy in Patients without Confirmed HCM

Type 1 error, or a high false positive rate, is a major concern for screening strategies for low-prevalence conditions like HCM. Furthermore, given that HCM is often underdiagnosed, it is important to evaluate the phenotypic characteristics of false positive cases. Therefore, among patients without CMR-confirmed HCM, we applied the model to 5,000 randomly selected ECGs recorded within 30 days of a TTE. These ECGs were taken from patients who had previously not been analyzed in development or evaluation sets. We extracted the end-diastolic interventricular septal wall thickness (IVSd), available as a continuous measure from TTEs. To evaluate the instances of model-positive screens in patients without CMR-confirmed HCM, we compared the IVSd measurements in false positive and true negative screens.

### Localization of Model Predictive Cues

To obtain a heatmap highlighting the portions of an ECG image that were important for predicting HCM, we used Gradient-weighted Class Activation Mapping (Grad-CAM) (Selvaraju R R, Cogswell M, Das A, Vedantam R, Parikh D, Batra D. Grad-CAM: Visual Explanations from Deep Networks via Gradient-based Localization. 2017 leee International Conference on Computer Vision (Iccv). 2017; 618-626). We calculated the gradients on the final stack of filters in our EfficientNet-B3 model for each prediction and performed a global average pooling of the gradients in each filter, emphasizing those that contributed to a prediction. We then multiplied these filters by their importance weights and combined them across filters to generate Grad-CAM heatmaps. Among the 25 positive cases with the most confident model predictions for HCM across ECG formats, we averaged class activation maps to determine the most important image areas for the prediction of HCM. We took an arithmetic mean across the heatmaps for a given image format and overlayed this average heatmap across a representative ECG before the conversion of the image to grayscale. The Grad-CAM intensities were converted from their original scale (0-1) to a color range using the jet colormap array in the Python library matplotlib, which was then overlaid on the original ECG image with an alpha of 0.3. The activation map, a 10×10 array, was upsampled to the original image size using the bilinear interpolation built into TensorFlow v2.8.0. We also evaluated the Grad-CAM for individual ECGs in the UK Biobank to evaluate the consistency of the information on individual examples.

### External Validation

We used data from the UK Biobank, under research application #71033, to pursue external validation of our model. UK Biobank represents the largest population-based cohort of 502,468 people in the United Kingdom with protocolized imaging and laboratory testing, along with linked electronic health records. Given the mean age at diagnosis for HCM among adults is 51±16 years, we evaluated our model among ECGs from participants where a majority were <70 years old, representing the population where an AI-ECG model may be used for identifying HCM. We used linked electronic health records for the participants to identify the presence of HCM diagnosis codes. In patients without HCM, we also used CMR-derived left-ventricular mass index (LVMI) to compare the characteristics of participants with a positive and negative AI-ECG screen for HCM. LVH was defined as LVMI >70 in men and LVMI >55 in women.

### Statistical Analysis

Categorical variables were reported as number (percentage, %), and continuous variables as mean (standard deviation [SD]) or median (interquartile range [IQR]), as appropriate. The model's performance was presented as area under the receiver operating characteristic curve (AUROC) and area under the precision-recall curve (AUPRC). The 95% confidence intervals (CI) for AUROC and AUPRC were calculated using DeLong's algorithm and bootstrapping with 1000 iterations, respectively. Furthermore, we reported sensitivity, specificity, positive predictive value (PPV), negative predictive value (NPV), and F1 score of the model at the model threshold for 90% sensitivity in the validation set. The statistical significance level was set at P<0.05. All statistical analyses were executed using Python 3.11.2 and R version 4.2.0.

### Results

### Study Population

We used data from 126,203 12-lead ECGs obtained from 68,109 patients at YNHH. The data from these patients were split into train, validation, and test sets at a patient level, as described in the methods. Individuals in the model development population (training and validation sets) had a median age of 63.2 years (IQR 51.2-74.1) at the time of ECG recording, and 33257 (49.6%) were women. Overall, 48475 (72.4%) were non-Hispanic White, 7978 (11.9%) were non-Hispanic Black, 6029 (9.0%) were Hispanic, 1300 (1.9%) were Asian, 376 (0.6%) were from other races, and information was missing for 2829 (4.2%). In the development population, there were 12,680 ECGs from 4745 patients with CMR-confirmation of HCM or echocardiographic parameters consistent with HCM (Table 2).

TABLE 2
Baseline characteristics of study population. Data presented as median [IQR] for age
and number (percent) for other variables.

| | Train | | Validation | | Test | |
|---|---|---|---|---|---|---|
| Characteristic | Patients | ECGs | Patients | ECGs | Patients | ECGs |
| Number | 66339 | 123640 | 648 | 913 | 1122 | 1650 |
| Sex; N(%) | | | | | | |
| Female | 33044 (49.8) | 58390 (47.2) | 213 (32.9) | 291 (31.9) | 372 (33.2) | 542 (32.8) |
| Male | 33013 (49.8) | 64675 (52.3) | 423 (65.3) | 606 (66.4) | 729 (65.0) | 1076 (65.2) |
| Missing | 282 (0.4) | 575 (0.5) | 12 (1.9) | 16 (1.8) | 21 (1.9) | 32 (1.9) |
| Age (years); | 63.3 | 64.1 | 58.7 | 60.5 | 56.9 | 58.4 |
| Median[IQR] | [51.2, 74.2] | [52.5, 75.0] | [52.8, 67.4] | [53.2, 67.6] | [44.8, 67.2] | [45.6, 68.4] |
| Race; N(%) | | | | | | |
| Asian | 1289 (1.9) | 2286 (1.8) | 11 (1.7) | 15 (1.6) | 27 (2.4) | 35 (2.1) |
| Black | 7892 (11.9) | 15984 (12.9) | 86 (13.3) | 122 (13.4) | 146 (13.0) | 216 (13.1) |
| Hispanic | 5964 (9.0) | 11297 (9.1) | 65 (10.0) | 84 (9.2) | 121 (10.8) | 170 (10.3) |
| White | 48038 (72.4) | 88711 (71.7) | 437 (67.4) | 628 (68.8) | 743 (66.2) | 1107 (67.1) |
| Other | 371 (0.6) | 702 (0.6) | 5 (0.8) | 7 (0.8) | 9 (0.8) | 14 (0.8) |
| Unknown | 2785 (4.2) | 4660 (3.8) | 44 (6.8) | 57 (6.2) | 76 (6.8) | 108 (6.5) |
| HCM; N(%) | 4715 (7.1) | 12597 (10.0) | 30 (4.6) | 83 (9.1) | 59 (5.3) | 150 (9.1) |
| CMR-Confirmed HCM | 505 (0.8) | 1887 (1.5) | 30 (4.6) | 83 (9.1) | 59 (5.3) | 150 (9.1) |
| CMR-Possible HCM | 79 (0.1) | 287 (0.2) | | | | |
| IVSd > 15 mm | 4131 (6.2) | 10190 (8.3) | | | | |

Abbreviations: CMR, cardiac magnetic resonance imaging; ECGs, electrocardiograms; HCM, hypertrophic cardiomyopathy; IVSd, interventricular septal thickness in diastole.

### Detection of HCM

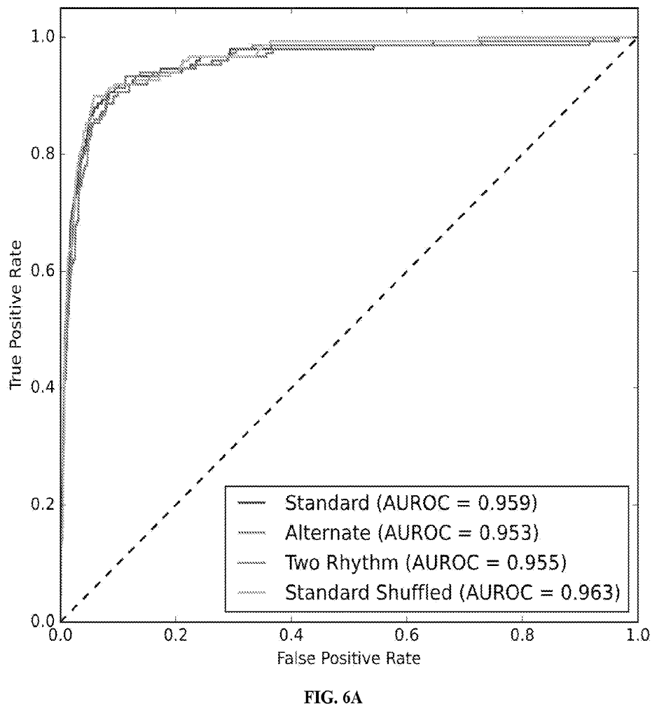

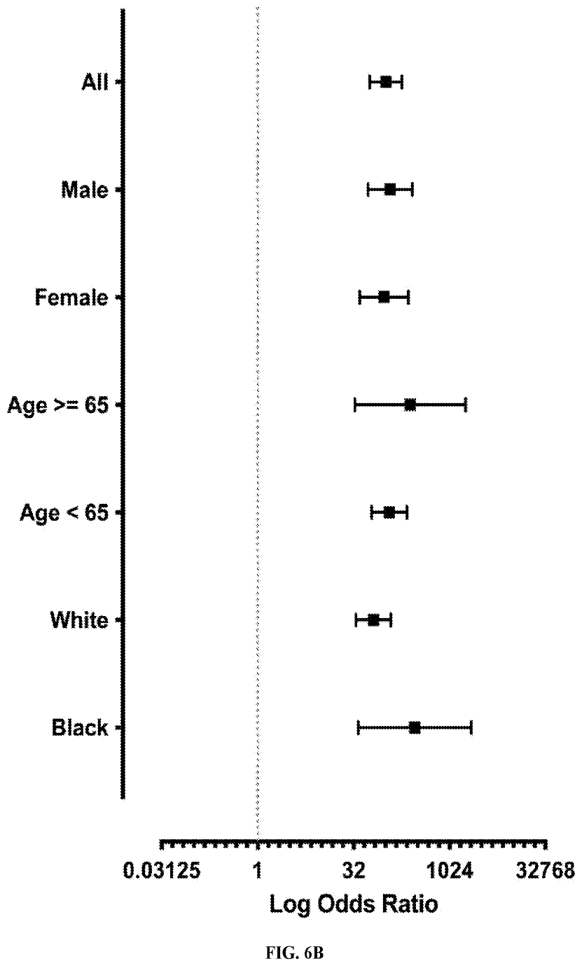

In the age- and sex-matched held-out test set comprising standard format images, the model for detecting HCM achieved an AUROC of 0.96 (FIGS. 6A-B). A probability threshold for predicting HCM was chosen based on a sensitivity of 0.90 or higher in the validation subset. With this threshold, the model had sensitivity and specificity of 0.91 and 0.91 in the held-out test set and PPV and NPV of 0.51 and 0.99, respectively. Overall, an ECG suggestive of HCM portended over 100-fold higher odds (OR 102.4, 95% CI, 57.4-182.6) of HCM (FIGS. 6A-B). The model's performance was comparable across subgroups of age, sex, and race (Table 3 and FIGS. 6A-B). The model performance was also comparable across the four original layouts of ECG images in the held-out set with an AUROC of 0.95-0.96 for detecting HCM. Sensitivity analyses demonstrated consistent model performance on ECGs without paced rhythms, atrial fibrillation and flutter, conduction disorders, and in the presence of LVH (Table 3).

TABLE 3
Performance of model on test images across demographic subgroups in the age sex
matched held-out test set.

| Labels | Number | PPV | NPV | Specificity | Sensitivity | AUROC | AUPRC | F1 Score |
|---|---|---|---|---|---|---|---|---|
| All | 1650 | 0.511 | 0.99 | 0.913 | 0.907 | 0.959 (0.941-0.977) | 0.754 (0.678-0.828) | 0.654 |
| Male | 1076 (65.2%) | 0.482 | 0.992 | 0.900 | 0.93 | 0.958 (0.933-0.983) | 0.743 (0.642-0.839) | 0.635 |
| Female | 542 (32.8%) | 0.589 | 0.985 | 0.940 | 0.86 | 0.966 (0.947-0.986) | 0.807 (0.691-0.901) | 0.699 |
| >=65 | 506 (30.7%) | 0.388 | 0.997 | 0.846 | 0.978 | 0.958 (0.938-0.978) | 0.633 (0.506-0.793) | 0.556 |
| <65 | 1144 (69.3%) | 0.607 | 0.987 | 0.943 | 0.875 | 0.96 (0.937-0.983) | 0.796 (0.705-0.87) | 0.717 |
| Hispanic | 170 (10.3%) | 0.462 | 1 | 0.957 | 1 | 1 (1-1) | 1 (1-1) | 0.632 |
| White | 1107 (67.1%) | 0.48 | 0.986 | 0.909 | 0.867 | 0.943 (0.917-0.97) | 0.673 (0.568-0.77) | 0.618 |
| Black | 216 (13.1%) | 0.661 | 0.993 | 0.882 | 0.975 | 0.986 (0.974-0.999) | 0.941 (0.871-0.986) | 0.788 |
| Asian | 35 (2.1%) | 0.4 | 1 | 0.897 | 1 | 0.983 (0.935-1) | 0.833 (0.333 - 1.0) | 0.571 |
| Other | 14 (0.8%) | 0 | 1 | 0.946 | — | — | — | — |
| Unknown | 108 (6.5%) | 0.444 | 1 | 0.928 | 1 | 0.971 (0.933-1) | 0.560 (0.25-1.0) | 0.615 |
| Paced ECGs | 28 (1.7%) | 0.409 | 1 | 0.316 | 1 | 0.655 (0.45-0.86) | 0.406 (0.234-0.716) | 0.581 |
| Not Paced ECGs | 1622 (9.8%) | 0.52 | 0.99 | 0.921 | 0.901 | 0.961 (0.943-0.98) | 0.803 (0.74-0.863) | 0.66 |
| A-Fib or Flutter | 75 (4.5%) | 0.324 | 1 | 0.641 | 1 | 0.886 (0.812-0.961) | 0.424 (0.237-0.708) | 0.489 |
| No A-Fib or | 1575 (95.5%) | 0.539 | 0.99 | 0.925 | 0.899 | 0.962 (0.943-0.981) | 0.795 (0.718-0.86) | 0.674 |
| Flutter | | | | | | | | |
| LBBB | 19 (1.2%) | 0.353 | 1 | 0.154 | 1 | 0.872 (0.706-1) | 0.719 (0.365-1.0) | 0.522 |
| No LBBB | 1600 (97.0%) | 0.523 | 0.992 | 0.921 | 0.920 | 0.963 (0.945-0.982) | 0.772 (0.692-0.841) | 0.667 |
| RBBB | 71 (4.3%) | 0.5 | 1 | 0.855 | 1 | 0.996 (0.988-1) | 0.98(0.904-1) | 0.667 |
| No RBBB | 1548 (93.8%) | 0.512 | 0.992 | 0.916 | 0.919 | 0.961 (0.943-0.98) | 0.758 (0.679-0.836) | 0.658 |
| LVH | 221 (13.4%) | 0.649 | 0.982 | 0.735 | 0.973 | 0.959 (0.934-0.984) | 0.916 (0.855-0.965) | 0.778 |
| No LVH | 1429 (86.6%) | 0.413 | 0.991 | 0.933 | 0.842 | 0.942 (0.91-0.974) | 0.543 (0.442-0.669) | 0.554 |

Abbreviations: PPV, positive predictive value; NPV, negative predictive value; AUROC, area under receiver operating characteristic curve; AUPRC, area under precision recall curve; A-Fib, atrial fibrillation; ECG, electrocardiogram; LBBB, left bundle branch block; RBBB, right bundle branch block; LVH, left ventricular hypertrophy.

### Evaluation of LVH Phenotype in Model-Predicted False Positives

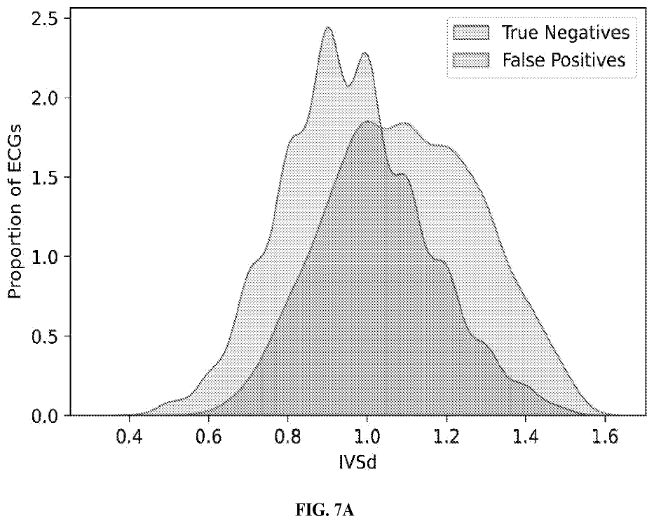

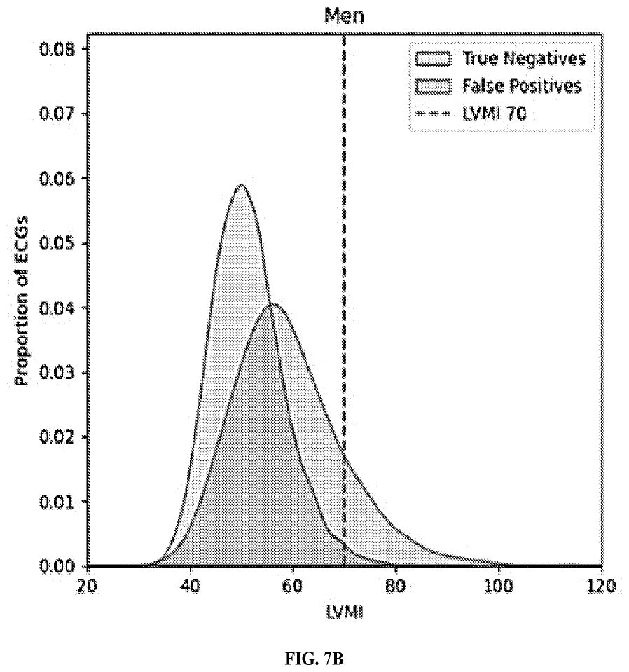

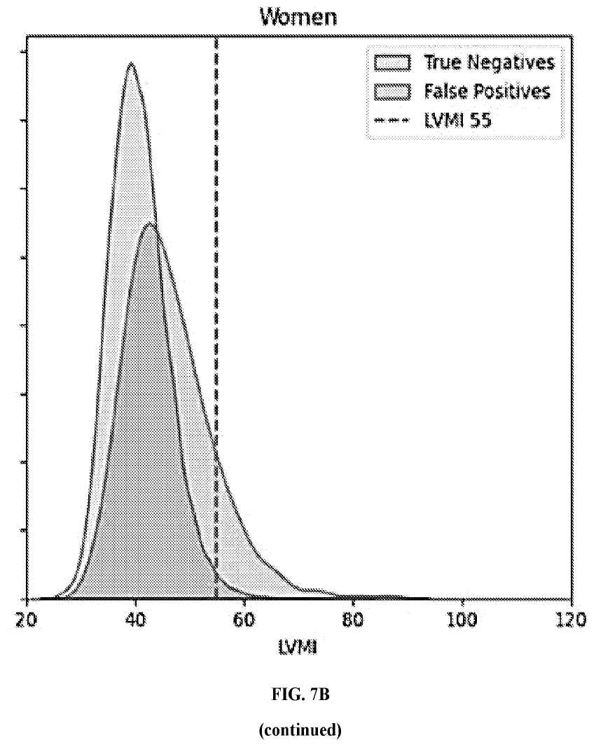

We applied the model to 5,000 randomly selected ECGs recorded within 30 days of a TTE in patients outside our development and held-out test sets. Of these, 647 (12.9%) were classified as false positives and 4,353 (87.1%) as true negatives. The median IVSd among the false-positive subset was 11 mm (IQR 9.7-12.4), compared with 9.5 mm (IQR 8.4-10.8) among false negatives (p<0.001) (FIG. 7A).

### Localization of Predictive Cues for HCM

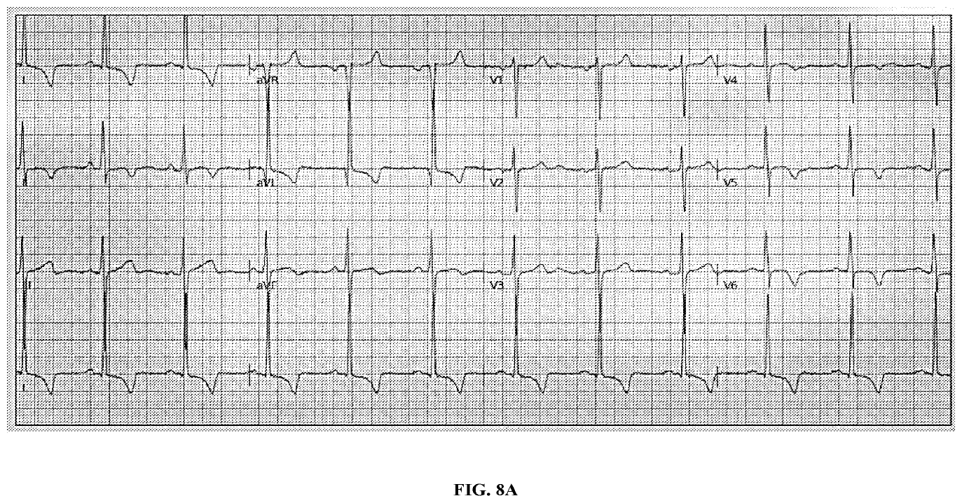

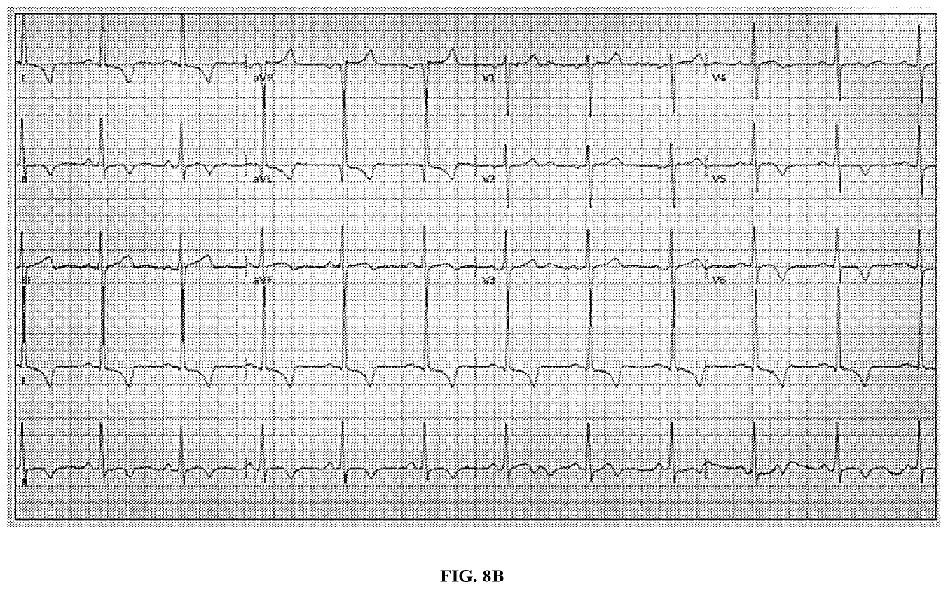

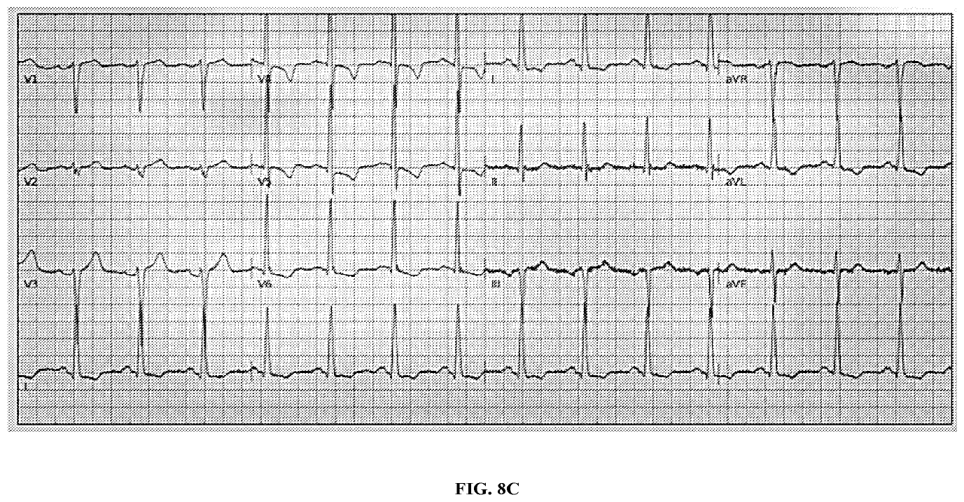

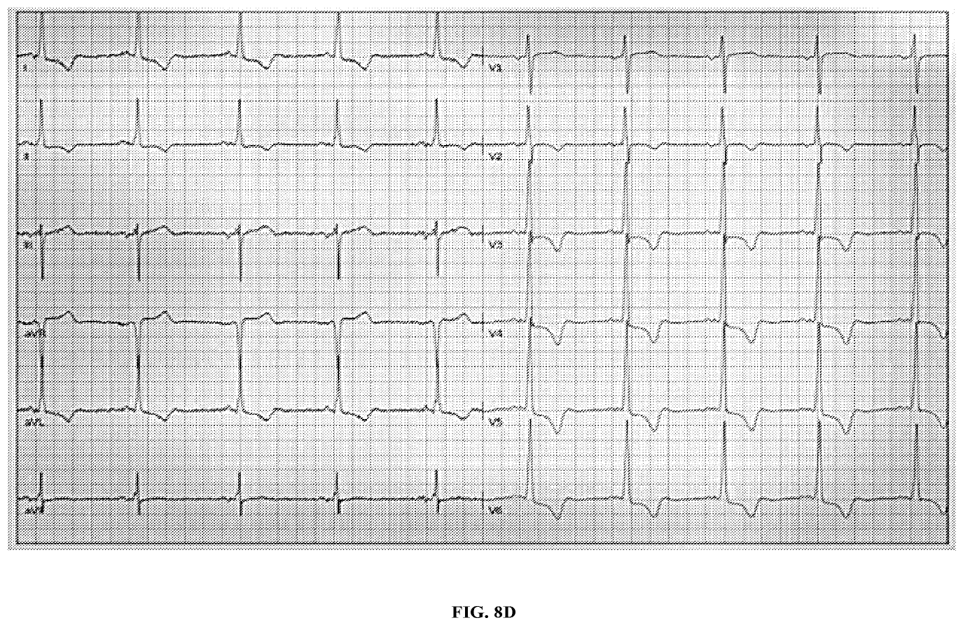

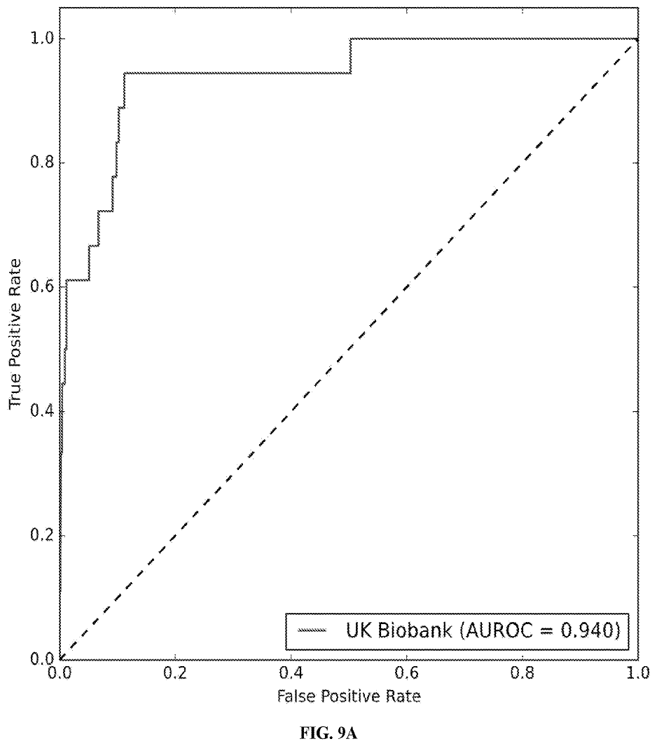

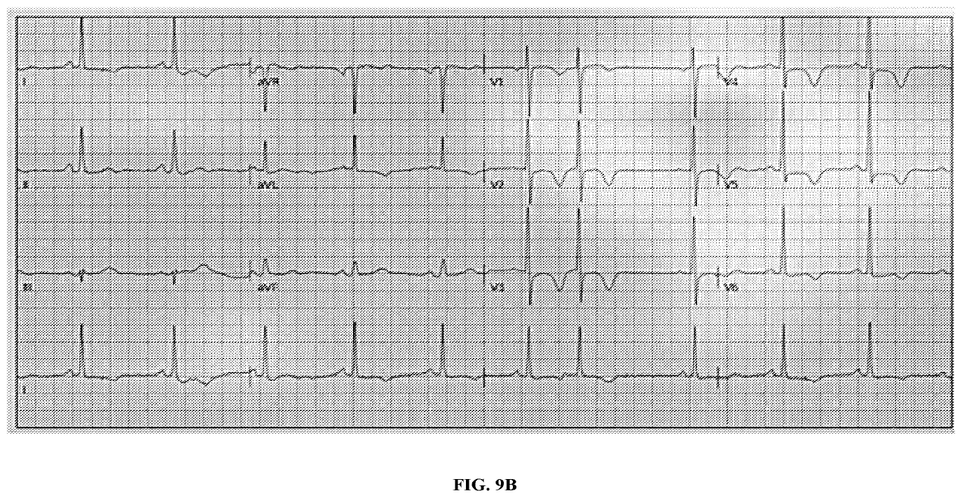

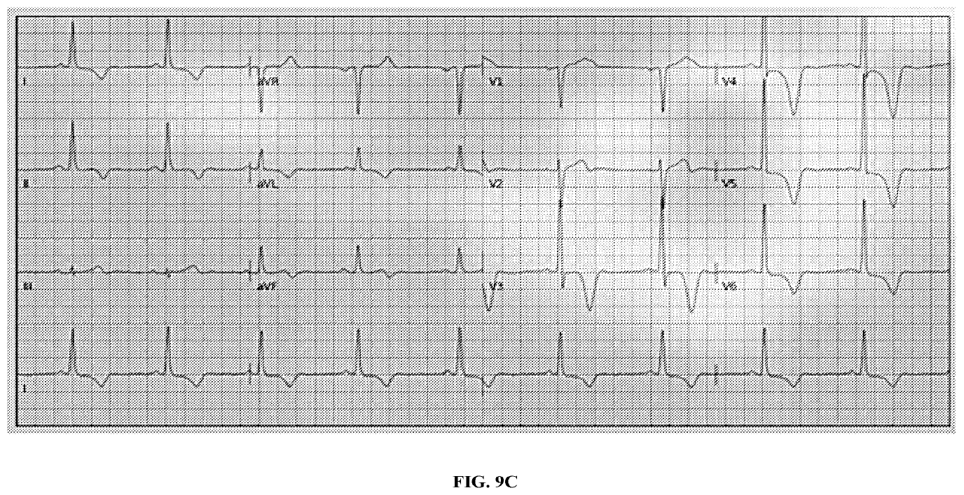

Class activation heatmaps of the 25 positive cases with the most confident model predictions for HCM prediction across four ECG layouts are presented in FIGS. 8A-D. For all four formats of images, the region corresponding to leads V4 and V5 were the most important areas for prediction of HCM. Representative images of Grad-CAM analysis in sampled individuals with positive screens in UK Biobank, the external validation site, showed similar patterns (FIGS. 9B-C).

### External Validation

We applied the model to the UK Biobank validation set, consisting of 32,885 ECGs from prospectively enrolled individuals, including 18 (0.05%) with an ICD code for HCM. The model had an AUROC of 0.94 (0.89-0.99) on these images (FIG. 9A), with a sensitivity of 0.61 and specificity of 0.96 at the threshold set in the development population. Of the 32,867 ECGs in this set without an HCM diagnosis, 32,859 were from individuals who had undergone cardiac MRIs. The model classified 1,322 (4.0%) as false positives and 31,537 (96.0%) as true negatives. Of the false positive screens, 153 (11.6%) had LVH, compared with 313 (1.0%) among true negative screens (FIG. 7B).

### Discussion

We developed and validated an automated deep learning model for identifying HCM solely from ECG images. The model is robust to variations in the layouts of ECG waveforms, making it suitable for implementation in various settings. Moreover, the model has excellent discrimination and sensitivity, representing characteristics ideal for screening. The model was developed and tested in a diverse population with high performance in subgroups of age, sex, and race. The model performance was consistent in the UK Biobank, a population-based cohort, despite a different, diagnosis code-based definition of HCM. An evaluation of the class-discriminating signals localized it to the anterior and lateral leads regardless of the ECG layout, topologically corresponding to the left ventricle. Therefore, an ECG image-based approach can represent a screening strategy for HCM, particularly in low-resource settings.

ECG-image-based deep learning models represent a novel application of AI that has the potential to improve clinical care and public health by offering a feasible modality for the early detection of HCM. Previously, various criteria have been proposed for the identification of HCM based on clinical interpretation of visible ECG features, such as abnormal Q or T waves, repolarization changes, or high QRS complexes. However, the accuracy of these clinical ECG-based criteria is limited, ranging from 55% to 80%. Moreover, the visible ECG abnormalities are not specific for HCM and are often present in various clinical conditions causing left ventricular hypertrophy, or rarely even as physiological variants. More recently, deep learning models utilizing raw ECG voltage signals have been proposed for the detection of HCM. While these models have excellent performance in internal validation, their development has often relied on diagnosis codes and visits to a specialty HCM clinic for the identification of HCM cases. These practices for recording diagnosis and administrative codes are often health-system-specific, and the use of diagnosis codes has been shown to misclassify up to one-third of HCM cases. Thus, to ensure the generalizability of deep learning models in diverse settings, it is critical to incorporate objective imaging-based biologic features for the identification of disease. Our use of CMR for the identification of disease represents a robust definition for identifying HCM, and enables the model to differentiate HCM from LVH-causing HCM mimics such as severe AS or hypertension. This is also demonstrated by consistently high discrimination of the model for HCM in the patient subgroup with increased septal wall thickness.

Using images to detect HCM signifies an advance over signal-based models, allowing for accessible implementation of a potential ECG-based screening approach. Digital or printed ECG images represent the most commonly available format, especially in low-resource settings. Moreover, ECG images are an interoperable data stream that is not tied to proprietary formats from specific ECG machine vendors, making them readily available to clinicians at the point-of-care. Currently, the guidelines for universal screening for HCM are equivocal, given the limited affordability of advanced cardiac imaging and the high number of false-positive and negative screens on clinically apparent ECG anomalies. However, an accurate and accessible approach to HCM diagnosis using AI can potentially make HCM screening economically viable, especially for people at elevated risk of sudden cardiac death, such as young athletes. Moreover, using ECG images in our model overcomes the implementation challenges of black box algorithms. The consistent localization of the risk-discriminative signals in anterior and lateral leads of ECG images, regardless of the lead location on printed images, indicates the left ventricular origin of the underlying pathology.

### Conclusions

We developed and validated a high-performing deep learning-based model that detects HCM from images of clinical 12-lead ECGs. This approach represents an accessible strategy for HCM screening, especially in low-resource settings.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of developing a deep learning model for detecting cardiovascular disorders from electrocardiographic images, the method comprising:
pre-training the nodes of a deep neural network to detect homologies of electrocardiogram (ECG) signatures in distinct ECG images from a single person; and
subsequently training the nodes on labeled ECG image examples of a cardiovascular disorder of interest.

2. The method of claim 1, wherein the pre-training includes training the nodes to determine when distinct ECGs belong to the same person.

3. The method of claim 2, wherein the training of the nodes to determine when distinct ECGs belong to the same person includes rewarding the model for identifying ECG images from the same individual as similar and penalizing the model for identifying ECG images from different individuals as similar.

4. The method of claim 3, wherein the pre-training reduces a contrastive loss function.

5. The method of claim 2, wherein the pre-training provides enhanced learning and detection of structural and functional abnormalities of the heart from any ECG image.

6. The method of claim 2, wherein, following the pre-training, the nodes recognize features that indicate whether distinct ECGs were obtained from a single person.

7. The method of claim 6, wherein the features are hidden features.

8. The method of claim 1, wherein the model adapts to detection of the cardiovascular disorder of interest during the training on the labeled examples.

9. The method of claim 1, wherein the pre-training is self-supervised and the subsequent training is supervised.

10. The method of claim 1, wherein the method includes training the nodes to recognize individual leads of the ECG regardless of their location on the ECG image.

11. The method of claim 1, wherein the method recognizes homologies of ECG signatures drawn from the same person.

12. The method of claim 1, wherein the labeled ECG image examples comprises a data set with a limited number of examples as compared to those for model development without pretraining procedures.

13. The method of claim 1, wherein the cardiovascular disorder of interest is a rare structural or functional heart disease.

14. The method of claim 1, wherein the model generates individualized phenotyping of disease in specific populations using a data set with a limited number of examples of patients with a disease as compared to those without pretraining procedure.

15. The method of claim 14, wherein the model identifies markers of cardiovascular disease using the limited data set.

16. The method of claim 1, wherein the cardiovascular disorder of interest is a common disorder.

17. The method of claim 16, wherein the model provides improved reliability for identification of physiological and pathological patterns on ECGs as compared to direct model development on the same data set.

18. The method of claim 1, further comprising detecting the cardiovascular disorder of interest by applying the model to ECG images from a subject.

19. The method of claim 18, wherein the model detects cardiovascular disorders from ECG images in a decentralized manner.

20. The method of claim 18, wherein the ECG images are format independent.

21. The method of claim 20, wherein the format independent ECG images are selected from the group comprising a screenshot of at least a portion of an ECG, a photograph of at least a portion of an ECG, a saved image file of at least a portion of an ECG, and combinations thereof.
